(19) United States
(12) Patent Application Publication
(10) Pub. No.: US 2024/0271086 A1
(43) Pub. Date: Aug. 15, 2024

(54) METHODS FOR ISOLATION AND CULTURE OF PRIMARY CELLS FROM AQUATIC ANIMAL TISSUES

(71) Applicant: CELLQUA INC, Seongnam (KR)

(72) Inventor: Sang Yoon LEE, Seongnam (KR)

(21) Appl. No.: 18/432,138

(22) Filed: Feb. 5, 2024

(30) Foreign Application Priority Data
Feb. 10, 2023 (KR) 10-2023-0018203

(51) Int. Cl. C12N 5/07; C12N 5/00; C12N 5/071; C12N 5/077

(52) U.S. Cl. CPC C12N 5/0601; C12N 5/0018; C12N 5/0658; C12N 5/067; C12N 5/0681; C12N 2500/12; C12N 2501/999; C12N 2509/10

(57) ABSTRACT

The present invention relates to a method for isolation and culture of primary cells from aquatic animal tissues, and specifically, to a method of culturing the tissue of an aquatic animal, comprising physically sectioning it without using a chemical reagent or enzyme and culturing it with a cell culture solution containing complex antibiotics.

### CROSS-REFERENCE TO RELATED APPLICATION

This application claims under 35 U.S.C. § 119(a) the benefit of Korean Patent Application No. 10-2023-0018203 filed on Feb. 10, 2023, the entire contents of which are incorporated herein by reference.

### REFERENCE TO AN ELECTRONIC SEQUENCE LISTING

The contents of the electronic sequence listing (40101-1004 Sequence Listing XML.xml; Size: 15,592 bytes; and Date of Creation: Mar. 15, 2024) is herein incorporated by reference in its entirety.

### BACKGROUND

### (a) Technical Field

The present invention relates to a method for isolation and culture of primary cells from aquatic animal tissues, and specifically, to a method of culturing the tissue of an aquatic animal, including physically sectioning it without using chemical reagents or enzymes and culturing it with a cell culture medium comprising complex antibiotics.

### (b) Background Art

Animal primary cells refer to cells isolated from tissue or blood created to carry out life activities. Even when cultured in vitro, the primary cells can represent the functions of cells in actual living tissue and generally have a lifespan, but there are differences depending on the species, tissue, and cell type. On average, the passage number of the primary cells used in research is limited to 2 to 5, but the standards also vary depending on the target organism and researcher. Additionally, as passage number increases, the primary cells may have extended lifespan or the ability to become immortal through mutations during cell proliferation and differentiation.

As much research has been conducted on culturing animal primary cells, various problems and technologies have been solved, but some problems still exist. Fundamental problems include the short lifespan of cells, lack of consistency in cell state, and animal welfare (ethics) issues used for cell isolation. Technical problems include the diversity of cell isolation methods depending on the species, the high cost of cell isolation and culture management, and limited production volume.

Research and industrial use of the primary cells of aquatic animals mostly involve research on the immunity of organisms used in aquaculture, development of cell lines for vaccine production, and production of proteins characteristically expressed in aquatic animals. The types and cases are very small.

Recently, the seriousness of animal welfare issues has been recognized worldwide, and primary cell research is increasing for physiological/genetic research on aquatic animals with high organism species diversity. However, the number of cells developed or distributed is still very limited, and in Korea, there is no aquatic animal cell bank, so there are no cases of distribution.

For the advancement of aquatic animal cell research, the most important thing is the establishment of technology for isolating and culturing tissue-specific primary cells from aquatic animal species. Until recently, research on isolating and culturing cells from aquatic animal tissues has been conducted, but the methods are diverse and complex, so there is no clear standard for what methodology is applied to obtain cells during actual experiments. In particular, aquatic invertebrates have been reported to be sensitive to some chemical reagents or enzymes used in cell isolation.

Therefore, the present invention is intended to introduce a method for isolating and culturing primary cells of various aquatic animal species without using chemical reagents or enzymes.

Thus, cells of various species and tissues of aquatic animals will be secured, and as the number of distribution cases increases, utilization and application in various industries will be possible. In particular, in the field of aquaculture, it is possible to develop products to improve growth speed, production, immunity, and quality, and it is expected that the present invention will be very useful in various fields, such as the production of various recombinant proteins and organism medicines for use in humans.

### SUMMARY OF THE DISCLOSURE

The technical problem to be achieved by the present invention is to provide a method of isolating and culturing primary cells from aquatic animal tissue without using chemical reagents.

However, the problem to be solved by the present invention is not limited to the problems mentioned above, and other problems not mentioned will be clearly understood by those skilled in the art from the following description.

An Example of the present invention provides a method for isolating and culturing primary cells from aquatic animal tissues, which includes the following steps of: isolating tissues from an aquatic animal; washing and disinfecting the isolated tissues; physically sectioning the washed and disinfected tissues; centrifuging the sectioned tissues to isolate cells from a portion of the sectioned tissues; and culturing the centrifuged tissues and cells with a cell culture solution comprising complex antibiotics, wherein the complex antibiotics comprise at least two of penicillin/streptomycin, amphotericin B, gentamycin, kanamycin, ampicillin and tetracyclin.

According to an Example of the present invention, the complex antibiotics may comprise penicillin/streptomycin, amphotericin B, gentamycin, kanamycin, ampicillin and tetracyclin.

According to an Example of the present invention, the culturing step may include a first culture with a cell culture solution comprising the complex antibiotics and then a second culture with a cell solution comprising a single antibiotic.

According to an Example of the present invention, the single antibiotic may be any one of penicillin/streptomycin, amphotericin B, gentamycin, kanamycin, ampicillin and tetracyclin.

According to an Example of the present invention, the first culture may be cultured for a period of 10 to 20 days or less, and the second culture may be cultured for a period of 5 days or more and 15 days or less.

According to an Example of the present invention, at least one of the cell culture solution comprising the complex antibiotics and the cell culture solution comprising the single antibiotic may comprise sea salt.

According to an Example of the present invention, the salt concentration of at least one of the cell culture solution comprising the complex antibiotics and the cell culture solution comprising the single antibiotic may be greater than 0 ppt and less than or equal to 30 ppt.

According to an Example of the present invention, the washing and disinfecting step may involve removing microorganisms by disinfecting for less than or equal to 1 minute using ethanol.

According to an Example of the present invention, the section may be cut using one or more types of scissors, scalpel and tweezers.

According to an Example of the present invention, the culturing step may be performed under a temperature condition of 10° C. or higher and 30° C. or lower.

An Example of the present invention provides a primary cell isolated and cultured using the method for isolation and culture of primary cells from aquatic animal tissues.

The method of isolating and culturing primary cells from aquatic animal tissues according to an Example of the present invention is effective in the survival and proliferation of aquatic animal tissues and cells without contamination by microorganisms and fungi in the water.

The method of isolating and culturing primary cells from aquatic animal tissues according to an Example of the present invention can improve aquatic animal ethical issues by replacing animal testing for aquatic animals living in various environments.

The aquatic animal tissues according to an Example of the present invention can be used in a wide range of industries, and in particular, in the food industry, they can be used in the field of developing aquatic cultured meat among alternative foods.

### BRIEF DESCRIPTION OF THE DRAWINGS

FIG. 1 is a schematic diagram illustrating the entire process of isolating and culturing primary cells from aquatic animal tissues.

FIG. 2 illustrates the electrophoresis results of the PCR product that amplified the cytochrome c oxidase I (COX1) gene in the mitochondria to confirm the species of the target organism used for cell isolation in Example 1 using a gel image analyzer.

FIG. 3A˜3C are the result of analyzing the COX1 gene base sequence in mitochondria to confirm the species of the target organism used for cell isolation in Example 1. The Anguilla japonica (a) COX1 gene registered in Genbank was designated as AJA_REF, and the Anguilla japonica COX1 gene used in the present invention was designated as AJA_EXP. The Litopenaeus vannamei (b) COX1 gene registered in Genbank was designated as LVA_REF, and the Litopenaeus vannamei COX1 gene used in the present invention was designated as LVA_EXP. The Todarodes pacificus (c) COX1 gene registered in Genbank was designated as TPA_REF, and the Todarodes pacificus COX1 gene used in the present invention was designated as TPA_EXP. Identical bases between the COX1 gene registered in Genbank and the COX1 gene used in the present invention are marked with *.

FIG. 4A˜4H are an enlarged photograph of the appearance of primary cells taken under a microscope on the 7th day (caudal fin, gonad, liver) and 13th day (muscle) after seeding, which were proliferating from the caudal fin (a, b), gonad (c, d), liver (e, f), muscle (g, h) tissue of Anguilla japonica in the Example.

FIG. 5A˜5F are an enlarged photograph of the appearance of primary cells taken under a microscope on the 1st day (gill, hepatopancreas, heart) and 9th day (muscle) after seeding, which were proliferating from the muscle (a, b), gill (c, d), hepatopancreas (e) and heart (f) tissue of Litopenaeus vannamei in the Example.

FIG. 6A˜6F are an enlarged photograph taken under a microscope of the appearance of primary cells on the 8th day (muscle, skin) and 11th day (heart) after seeding, which were proliferating from the muscle (a, b), heart (c, d), and skin (e, f) tissue of squid in the Example.

### DETAILED DESCRIPTION

Globally, ESG (environmental, social and corporate governance) trends are shifting from climate crisis to organism diversity. Research on animals must be conducted after obtaining animal welfare/ethics approval before experimentation. When animal testing cannot be replaced, animals are inevitably used. In this case, one of the methods that can replace animal testing depends on the distribution of cells. This means that all animal testing cannot be replaced with cells, but the use of animals can be reduced if at least preliminary testing can be performed with cells.

Most current research has been focused on humans, but recently, research on organisms themselves is increasing, and aquatic animals are also becoming the target. Nevertheless, there are currently no institutions in Korea that can receive distribution of aquatic animal cells, so the number of distributions is 0, and overseas institutions inevitably make distribution, but this is also limited to cells of a very small type of species and tissue.

This is due to the lack of infrastructure to conduct aquatic animal cell research, and therefore, the technology or method for isolating cells from aquatic animals is also not standardized. Accordingly, the present invention intends to introduce a method for isolating and culturing primary cells from aquatic animal tissues of various species among the various problems mentioned above. Therefore, this includes information on the basic medium composition, types of additives, primary cell isolation method, and primary cell culture method used throughout the entire process for isolating primary cells.

Herein, when a part “includes” a certain component, this means that it may further include other components rather than excluding other components unless specifically stated to the contrary.

Herein, “A and/or B” means “A and B, or A or B”.

Hereinafter, the present invention will be described in more detail.

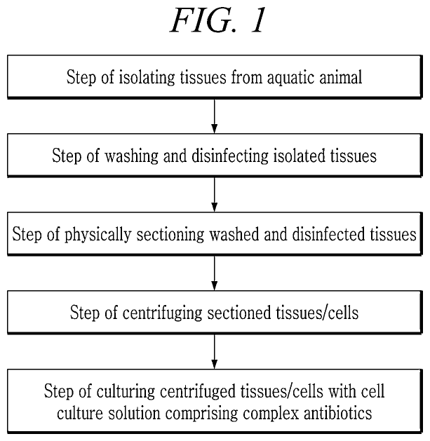

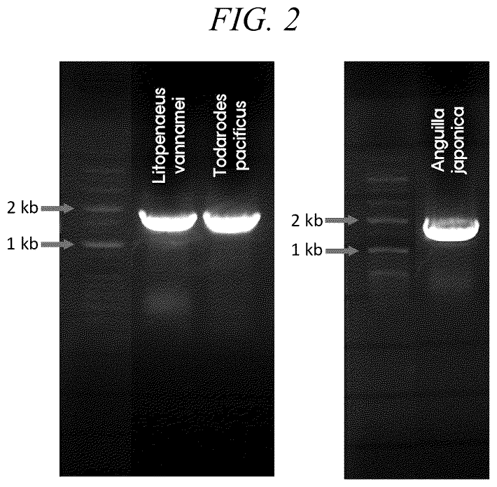

FIG. 1 shows a schematic diagram illustrating the entire process of isolating and culturing primary cells from aquatic animal tissues.

As shown in FIG. 1, the method for isolation and culture of primary cells from aquatic animal tissues includes the steps of: isolating tissues from an aquatic animal; washing and disinfecting the isolated tissues; physically sectioning the washed and disinfected tissues; centrifuging the sectioned tissues to isolate cells from a portion of the sectioned tissues; and culturing the centrifuged tissues and cells with a cell culture solution comprising complex antibiotics.

In addition, the complex antibiotics comprise at least two of penicillin/streptomycin, amphotericin B, gentamycin, kanamycin, ampicillin and tetracyclin. In the case of aquatic animals with high species diversity, the possibility of contamination with various types of microorganisms is high as their habitats are diverse. Therefore, if at least two types of antibiotics are used, contamination with various types of microorganisms can be prevented. On the other hand, when using a single antibiotic, the effect during cell culture may be low due to microorganism contamination.

The concentration of the complex antibiotics may be 0.5 to 2.0% (v/v), and different concentrations may be applied depending on the type of antibiotic.

Before culturing the aquatic animal tissues and cells, the species of the target animal may be confirmed.

FIG. 2 illustrates the electrophoresis results of the PCR product photographed using a gel image analyzer that amplified the cytochrome c oxidase I (COX1) gene in the mitochondria to confirm the species of the target organism used for cell isolation according to an Example of the present invention.

In an Example of the present invention, the PCR reaction conditions for amplifying the mitochondrial cytochrome c oxidase subunit I (COX1) gene in a genetic test performed to determine the species of an organism may differ depending on the vertebrates and invertebrates or species. Specifically, the COX1 gene was amplified under conditions of, for vertebrates, 95° C., 120 seconds (pre-denaturation)/95° C., 20 seconds (denaturing)/55° C., 30 seconds (annealing)/72° C., 90 seconds (extension)/72° C. and 300 seconds (final-extension), and for invertebrates, 95° C., 120 seconds (pre-denaturation)/95° C., 20 seconds (denaturing)/52° C., 30 seconds (annealing)/60° C., 90 seconds (extension)/60° C. and 300 seconds (final-extension). Under these conditions, the extension reaction time may be changed depending on the length of the COX1 gene or the length of the region to be amplified, and the annealing reaction temperature and time may be changed depending on the PCR primer composition used.

The PCR reaction conditions for the mitochondrial gene COX1 according to the present invention can solve the problem of PCR amplification failure due to secondary structure problems of the base sequence in vertebrates or invertebrates.

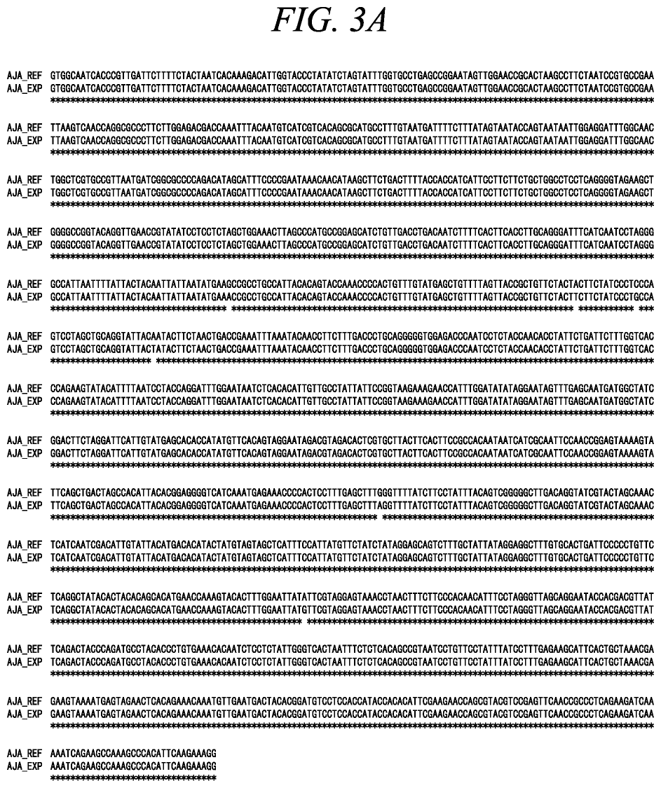

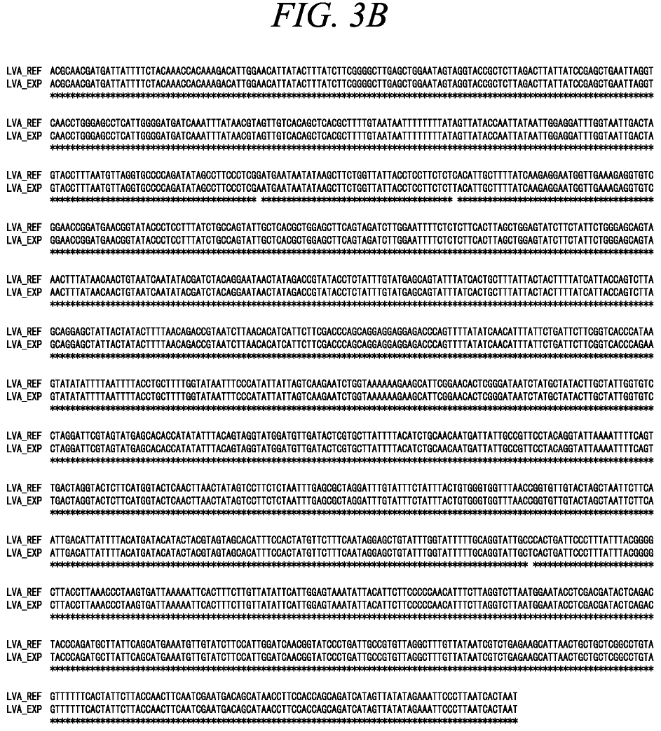

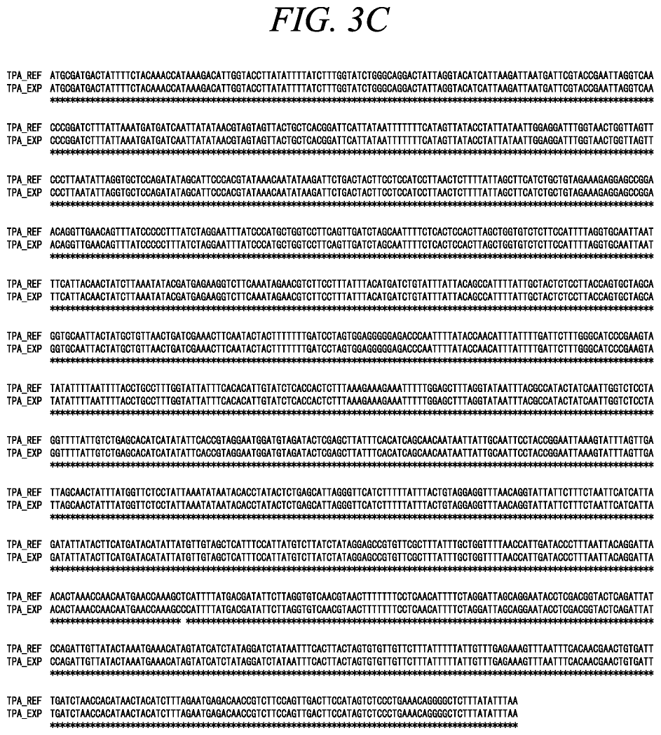

FIG. 3 is the result of analyzing the COX1 gene base sequence in mitochondria to confirm the species of the target organism used for cell isolation according an Example of the present invention.

The Anguilla japonica (a) COX1 gene registered in Genbank was designated as AJA_REF, and the Anguilla japonica COX1 gene used in the present invention was designated as AJA_EXP. The Litopenaeus vannamei (b) COX1 gene registered in Genbank was designated as LVA_REF, and the Litopenaeus vannamei COX1 gene used in the present invention was designated as LVA_EXP. The Todarodes pacificus (c) COX1 gene registered in Genbank was designated as TPA_REF, and the Todarodes pacificus COX1 gene used in the present invention was designated as TPA_EXP. Identical bases between the COX1 gene registered in Genbank and the COX1 gene used in the present invention are marked with *.

According to an Example of the present invention, aquatic animal tissues and cells are cultured after confirming the target organism's species.

Meanwhile, in the case of aquatic animals with high species diversity, the possibility of contamination by various types of microorganisms is high due to the variety of habitat environments, so using complex antibiotics rather than single antibiotics is more effective in preventing cell contamination than using species-specific selective antibiotics.

Therefore, according to an Example of the present invention, the complex antibiotics may include penicillin/streptomycin, amphotericin B, gentamycin, kanamycin, ampicillin, and tetracyclin. In the case of aquatic animals with high species diversity, the possibility of contamination by various types of microorganisms is high as their habitats are diverse. Therefore, if all antibiotics listed above are used, contamination by various types of microorganisms can be prevented. On the other hand, when using a single antibiotic, the effect during cell culture may be low due to microorganism contamination.

When selecting a type of antibiotic, there are many variables in microorganism contamination because the distribution of microorganisms varies depending on the habitat and rearing environment of the target aquatic animal from which cells are to be obtained, and the possibility of microorganism contamination also varies depending on the tissue cleaning method.

The most widely used antibiotics in cell culture, penicillin/streptomycin alone, had a contamination probability of 50% or more when isolating and culturing primary cells of aquatic animals.

The concentration of the complex antibiotics may be 0.5 to 2.0% (v/v), and different concentrations may be applied depending on the type of antibiotic.

According to an Example of the present invention, the culturing step may comprise a first culture with a cell culture solution comprising the complex antibiotics and then a second culture with a cell culture solution comprising a single antibiotic.

According to an Example of the present invention, the single antibiotic may be any one of penicillin/streptomycin, amphotericin B, gentamycin, kanamycin, ampicillin and tetracyclin.

Specifically, the single antibiotic refers to a single type of antibiotic such as amphotericin B, gentamycin, kanamycin, ampicillin and tetracyclin, as well as the single use of a “penicillin/streptomycin antibiotic solution”. The “penicillin/streptomycin antibiotic solution” is a mixture of penicillin G and streptomycin, which is the most widely used antibiotic in cell culture media and plays a role in preventing contamination during cell culture.

By selecting the single antibiotic from the above, the cost incurred in the culturing process can be minimized.

In addition, the first culture may be cultured for a period of 10 to 20 days, and the second culture may be cultured for a period of 5 to 15 days.

The first culture may mean that when culturing aquatic animal cells, the cell culture solution comprising complex antibiotics may be replaced every day for at least 3 days after seeding, and then the cell culture solution comprising complex antibiotics may be replaced every 2 to 4 days for 1 to 2 weeks.

The first culture period may be a total of 10 to 20 days, including the initial 3 days of daily replacement of the cell culture solution comprising complex antibiotics. If the period of the first culture does not satisfy the above period, the effect of cell culture may be low because the high risk of contamination with various types of microorganisms due to the diverse habitat environments of aquatic animals with high species diversity.

After the first culture period has elapsed, the first culture may be repeated two or more times depending on the cell culture state of the aquatic animal. In addition, after the first culture has elapsed, the cell culture solution comprising complex antibiotics can be replaced at intervals of 2 to 4 days depending on the culture state of the tissue and cells of the aquatic animal.

The second culture may mean that the cell culture solution comprising a single antibiotic can be replaced at intervals of 2 to 4 days after the first culture is completed.

The second culture period may be 5 to 15 days after the first culture is completed. After the second culture period has elapsed, the first culture or the second culture may be repeated depending on the culture state of the tissues and cells of the aquatic animal.

According to the Example of the present invention, at least one of the cell culture solution comprising the complex antibiotics and the cell culture solution comprising the single antibiotic may comprise sea salt.

Specifically, the medium composition for cell culture may include sea salt to adjust the osmotic pressure inside and outside the cell in addition to Leibovitz's L-15 component. In the case of Leibovitz's L-15, even if it does not include other sea salts, its basic salinity is about 8%.

According to the Example of the present invention, the salt concentration of at least one of the cell culture solution comprising the complex antibiotics and the cell culture solution comprising the single antibiotic may be greater than 0 ppt and less than or equal to 30 ppt.

Specifically, the medium for aquatic animal cell culture may comprise seawater prepared with different concentrations of sea salt depending on the species in order to maintain osmotic stability inside and outside the cells.

More specifically, cell-base medium can be prepared with a salinity of 5 to 10 ppt for

Anguilla japonica and 15 to 25 ppt for Litopenaeus vannamei and Todarodes pacificus. However, in the case of organisms that can survive even at various salinities, such as Anguilla japonica and Litopenaeus vannamei, the salinity of the cell-base medium can be set in various ways depending on the habitat or rearing environment conditions before tissue isolation.

According to the Example of the present invention, other types of basal media other than Leibovitz's L-15 can also be used. Accordingly, the type of additional basal medium may be any one selected from basal media for cell culture, such as BME (Basal Medium Eagle), DMEM (Dulbecco's Modified Eagle's Medium), MEM (Minimal essential Medium), M 199 (Medium 199) and RPMI 1640 (Roswell Park Memorial Institute 1640). In addition, when using a cell culture solution using a basal medium other than Leibovitz's L-15, the appropriate culture temperature must be the same as the conditions mentioned in the Example, but the CO₂ concentration must be maintained at 5%.

According to an Example of the present invention, in the step of isolating tissue from the aquatic animal, the tissue may be isolated by dissecting the aquatic animal after anesthetizing it. The anesthesia step can be performed with clove oil at a concentration of 90 to 110 ppm or at low water temperature (0° C. or lower). Afterwards, the target tissue (approximately 0.05 g or more) is isolated.

In the step of washing and disinfecting the isolated tissue, to prevent contamination of microorganisms, diluted ethanol is reacted for more than 0 minutes and less than or equal to 1 minute when washing the tissue, the separated tissue is washed 3 to 5 times with 90 to 110 ppm phosphate-buffered saline (PBS) or sterilized seawater (or sea salt), which has the same salinity as the cell base medium used, and then the ethanol can be removed.

In the case of invertebrates, due to their sensitivity to chemical reagents and enzymes, if the diluted ethanol reaction time is longer than 1 minute, the effect on cell attachment and proliferation may be further reduced, so it is important to disinfect and wash within the above time range.

The step of physically sectioning the washed and disinfected tissue is characterized by not adding typical chemical reagents or enzymes such as trypsin and collagenase.

The section may be sectioned using one or more types of scissors, scalpel, and tweezers. Preferably, the washed and disinfected tissue can be placed in a tube and cut thinly with sterilized dissection scissors. However, any other means capable of sectioning in accordance with the present invention may be included for those skilled in the art.

The present invention intends to present a method that can be applied to a wide range of aquatic animal species. Even within aquatic animals, treatment with chemical reagents or enzymes may be effective in isolating and proliferating primary cells in vertebrates, but in invertebrates, the success rate in isolating and proliferating primary cells is low when treated with chemical reagents or enzymes. Therefore, the method proposed in the present invention can be effective in cell separation and proliferation in both vertebrates and invertebrates.

The step of centrifuging the sectioned tissue to isolate cells from a portion of the sectioned tissue includes adding 1 mL to 3 mL of customized cell culture solution to the sectioned tissue and cells, mixing them, and then centrifuging (1500 rpm to 2500 rpm, 10 minutes to 30 minutes) to remove all of the supernatant.

The tissue refers to a collection of cells of the same type, and the cell culture solution used for centrifugation may be the cell culture solution prepared above and comprising complex antibiotics.

According to an Example of the present invention, the culturing step may be performed under temperature conditions of 10° C. or more and 30° C. or less. Specifically, the temperature can be between 22° C. or more and 28° C. or less for Anguilla japonica and Litopenaeus vannamei, and it can be between 15° C. or more and 22° C. or less for Todarodes pacificus. If the above temperature range is satisfied, cell culture can be more effective as the environment is similar to the organism's habitat.

According to an Example of the present invention, aquatic animal primary cells isolated and cultured using the above method for isolation and culture of primary cells from aquatic animal tissues are included.

The aquatic animal tissues are based on a minimum weight of 0.05 g or more, and the container used for tissue separation may be any one selected from a 1.5 mL (tissue weight of approximately 0.05 g) or 5.0 mL (tissue weight of approximately 0.2 g) tube that is less exposed to contamination and is easy to section with dissecting scissors. In addition, the culture vessel and tissue/cell amount used for seeding after sectioning may be any one selected from a T-25 flask (tissue/cell weight of approximately 0.025 g), a T-75 flask (tissue/cell weight of approximately 0.075 g) or a 90 mm cell culture dish (tissue/cell weight of approximately 0.05 g) with a surface treatment capable of cell attachment.

Hereinafter, the present invention will be described in detail using the Example to explain the present invention specifically. However, the Examples according to the present invention may be modified into various other forms, and the scope of the present invention is not to be construed as being limited to the Examples described below. The Examples in this specification are provided to more completely explain the present invention to those with average knowledge in the art.

### Preparation Example: Preparation of Cell Basal Medium

The basal medium for culturing aquatic animal primary cells used in the present invention was Leibovitz's L-15, and was prepared with different salinities depending on the species of aquatic animal.

Sea salt (Blue Treasure company) was used to control salinity, and powdered form of L-15 medium (Sigma-Aldrich company) was used. Sea salt was dissolved in tertiary distilled water and sterilized by heat at 115 to 125° C. for 10 to 20 minutes before use. The powdered L-15 medium was added to sterilized seawater and used after measuring salinity. Finally, the final cell base medium was prepared after filtration and sterilization using a 0.1 to 0.22 um filter (GVS company).

Among the experimental models used in this study, cell base medium was produced with a salinity of 5 to 10 ppt for Anguilla japonica and 15 to 25 ppt for Litopenaeus vannamei and Todarodes pacificus.

### Experimental Example: Genetic Testing and Analysis to Identify Organism Species

Before securing primary cells, genetic testing was performed using COX1 in mitochondria, which is used as a genetic marker, to distinguish the species of the target organism. First, genomic DNA was extracted and purified from Anguilla japonica, Litopenaeus vannamei, and Todarodes pacificus (Reference: Lee, S. Y., Lee, H. J., & Kim, Y. K. (2019). Comparative analysis of complete mitochondrial genomes with Cerithioidea and molecular phylogeny of the freshwater snail, Semisulcospira gottschei (Caenogastropoda, Cerithioidea). International journal of biological macromolecules, 135, 1193-1201.). Afterwards, for gene amplification, primers were designed as shown in Table 1 below based on the mitochondrial genome of Anguilla japonica (Genbank assession no: NC_002707.2), Litopenaeus vannamei (Genbank assession no: NC_009626.1), Todarodes pacificus (Genbank assession no: NC_006354.1). Table 1 below is a table showing PCR reaction primer information used in the Experimental Example of the present invention. Specifically, the following manufactured primers were used for gene amplification: AJA_mtCOX1_1F and AJA_mtCOX1_1R for Anguilla japonica, LVA_mtCOX1_1F and LVA_mtCOX1_1R for Litopenaeus vannamei, and TPA_mtCOX1_1F and TPA_mtCOX1_1R for Todarodes pacificus.

TABLE 1

| | | | | GC | |
|---|---|---|---|---|---|
| | | Base Sequence | Length | Content | TM value |
| Organism | Primer Name | (5′-3′) | (bp) | (%) | (° C.) |
| Anguilla | AJA_mtCOX1_1F | GTGTTATACACCACAGAGCT | 20 | 45 | 55.2 |
| japonica | AJA_mtCOX1_2F | GAGCAATGATGGCTATCGGA | 20 | 50 | 58.9 |
| | AJA_mtCOX1_1R | AAAGTGGCAGAGTGGTTATG | 20 | 45 | 56.7 |
| | AJA_mtCOX1_2R | GGTTCATGTGCTGTGTAGTG | 20 | 50 | 57.6 |
| Litopenaeus | LVA_mtCOX1_1F | CCACTACAAAGCCATGATAA | 20 | 40 | 53.6 |
| vannamei | LVA_mtCOX1_2F | AAGAAGCATTCGGAACACTCG | 21 | 48 | 59.6 |
| | LVA_mtCOX1_1R | AACCTAAGTGACCTCATGTT | 20 | 40 | 55.1 |
| | LVA_mtCOX1_2R | AAGCACGAGTATCAACATCC | 20 | 46 | 56.1 |
| Todarodes | TPA_mtCOX1_1F | GAGTGAAATCAAGGATCCCT | 20 | 45 | 57.1 |
| pacificus | TPA_mtCOX1_2F | TATCCCATGCTGGTCCTTCA | 20 | 50 | 61.2 |
| | TPA_mtCOX1_1R | AAAGGGGAACAAGCATCTTG | 20 | 45 | 58 |
| | TPA_mtCOX1_2R | ACCTGTTAAACCTCCTACAG | 20 | 45 | 56 |

Table 2 below shows the PCR reaction conditions used in an Experimental Example of the present invention. The PCR (ABI company) reaction temperature and time applied during gene amplification are shown in Table 2 below. PCR reaction materials were used by adding genomic DNA (500 ng or less) and 10 pmole each of PCR forward and reverse primers to AccuPower® ProFi Taq PCR PreMix 20 (Bioneer company) and adjusting to 20 ul with tertiary distilled water.

The PCR amplification product was confirmed by electrophoresis using 0.7% agarose, and as a result, the PCR product was confirmed to be a band of the same size as expected, as shown in FIG. 2. The amplification product was purified using the DokDo-Prep Gel/PCR Purification Kit (ELPIS-BIOTECH). Finally, the purified product was secured by requesting Sanger sequencing (MACROGEN company) to secure the base sequence. Sequencing was performed using all four types of primers for each aquatic animal as shown in Table 1 above.

TABLE 2

| | | Pre- | | | | Final- |
|---|---|---|---|---|---|---|
| | PCR | denaturation | Denaturing | Annealing | Extension | extension |
| Organism | cycling | (° C./seconds) | (° C./seconds) | (° C./seconds) | (° C./seconds) | (° C./seconds) |
| Anguilla japonica | 35 | 95 | 95 | 55 | 72 | 72 |
| | | 120 | 20 | 30 | 90 | 300 |
| Litopenaeus | 35 | 95 | 95 | 52 | 60 | 60 |
| vannamei, | | 120 | 20 | 30 | 90 | 300 |
| Todarodes | | | | | | |
| pacificus | | | | | | |

Using genetic analysis, the genetic information registered in the existing Genbank and the genetic information obtained through this experiment were compared and analyzed. The Anguilla japonica COX1 gene was 1593 bp, the Litopenaeus vannamei COX1 gene was 1533 bp, and the Todarodes pacificus COX1 gene was 1533 bp. As shown in FIG. 3, the subjects used in the Experimental Example of the present invention were confirmed to be the same species as the base sequences which were more than 99% identical to the COX1 genes of Anguilla japonica, Litopenaeus vannamei, and Todarodes pacificus already registered in Genbank.

### Example: Isolation and Culture of Primary Cells From Aquatic Animal Tissues

The use of aquatic animals was reviewed by the Animal Experiment Ethics Committee within Cellqua Co., Ltd., and tissue was isolated from the aquatic animals.

To isolate aquatic animal tissues, anesthesia was performed with 90 to 110 ppm of clove oil or low water temperature (0° C. or lower). After isolating the target tissue (approximately 0.05 g or more), it was washed 3 to 5 times with sterilized washing solution (PBS or seawater with the same salinity as the cell base medium). The washed tissue was reacted with diluted ethanol for less than or equal to 1 minute for disinfection and removal of microorganisms and then washed 3 to 5 times with a sterilized washing solution.

The tissue that had been washed and disinfected was placed in a 1.5 mL (tissue weight of approximately 0.05 g) or 5.0 mL (tissue weight of approximately 0.2 g) tube (SPL company) and thinly cut with sterilized dissection scissors. Afterwards, 1 to 3 mL of cell culture solution containing complex antibiotics [1 to 2% (v/v) penicillin/streptomycin, 0.5 to 2% (v/v) amphotericin B, 0.5 to 2% (v/v) gentamycin, 0.5 to 2% (v/v) kanamycin, 0.5 to 2% (v/v) ampicillin and 0.5 to 2% (v/v) tetracyclin] and 5 to 20% (v/v) fetal bovine serum (FBS) was added thereto and mixed. After centrifugation (2000 rpm, 10 to 30 minutes), all supernatant was removed.

Centrifuged tissues or cells were seeded in T-25 or T-75 flasks (SPL company) or 90 mm cell culture dishes (SPL company) with surface treatment for cell attachment. [T-25 flask (tissue/cell weight of approximately 0.025 g), T-75 flask (tissue/cell weight of approximately 0.075 g) or 90 mm cell culture dish (tissue/cell weight of approximately 0.05 g)]. Cell culture solution containing complex antibiotics and fetal bovine serum (FBS) was added thereto and cultured at an appropriate temperature [Anguilla japonica and Litopenaeus vannamei (22 to 28° C.), Todarodes pacificus (15 to 22° C.)].

After seeding, the cell culture solution containing the complex antibiotics was replaced every day for at least 3 days, and the cell culture solution containing the complex antibiotics was replaced every 2 to 4 days for the next 1 to 2 weeks (First culture refers to the entire period of culturing with cell culture solution containing complex antibiotics after seeding). Afterwards, cell growth was observed while changing the cell culture solution containing complex or single antibiotic (1% (v/v) penicillin/streptomycin) every 2 to 4 days (Second culture refers to the entire period of culturing with cell culture solution containing a single antibiotic after the first culture).

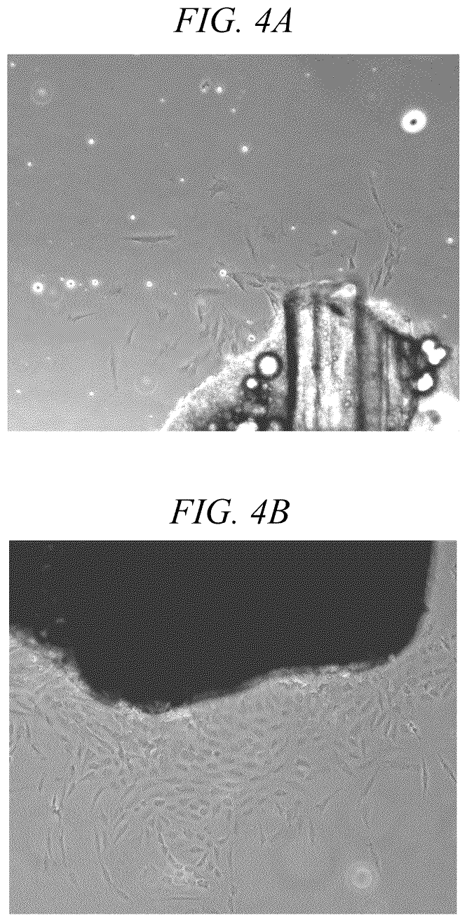

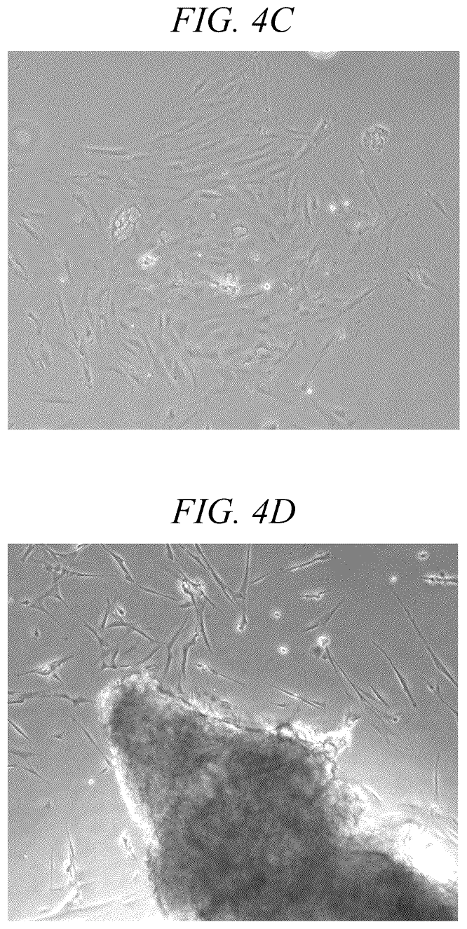

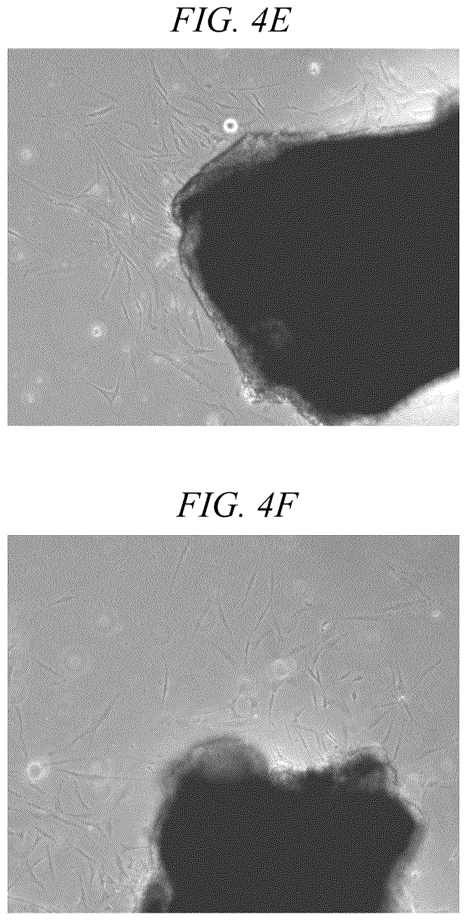

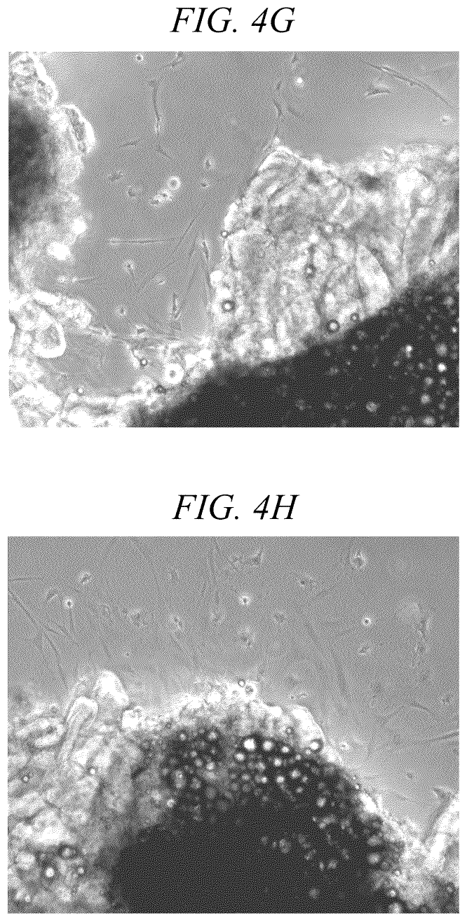

FIG. 4 is an enlarged photograph of the appearance of primary cells taken under a microscope (magnification: 100×, product name: AE31E, manufacturer: Motic company) on the 7th day (caudal fin, gonad, liver) and 13th day (muscle) after seeding, which were proliferating from the caudal fin (a, b), gonad (c, d), liver (e, f), muscle (g, h) tissue of Anguilla japonica according to the present invention.

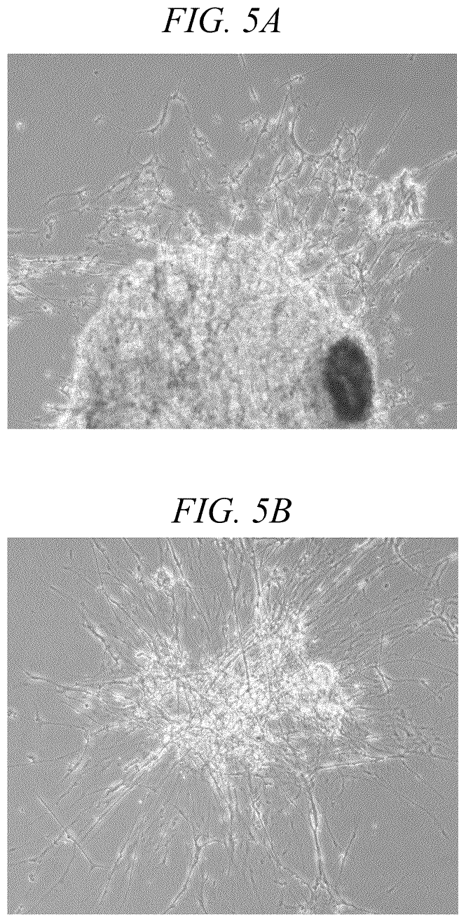

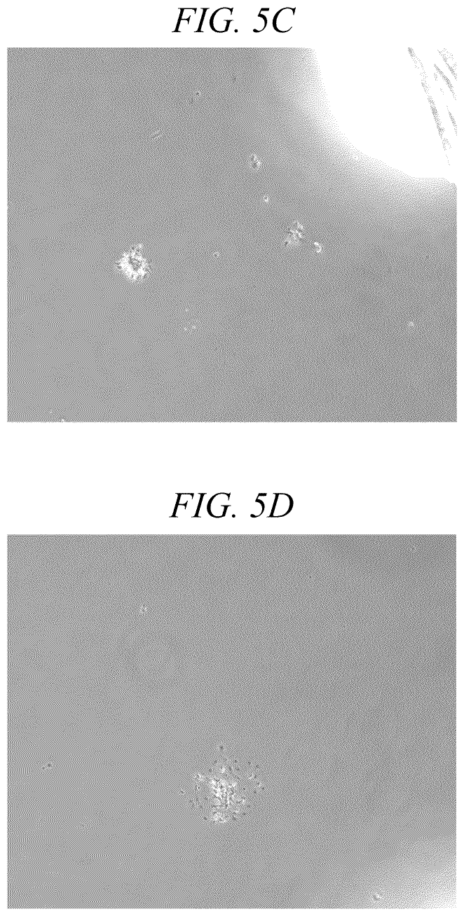

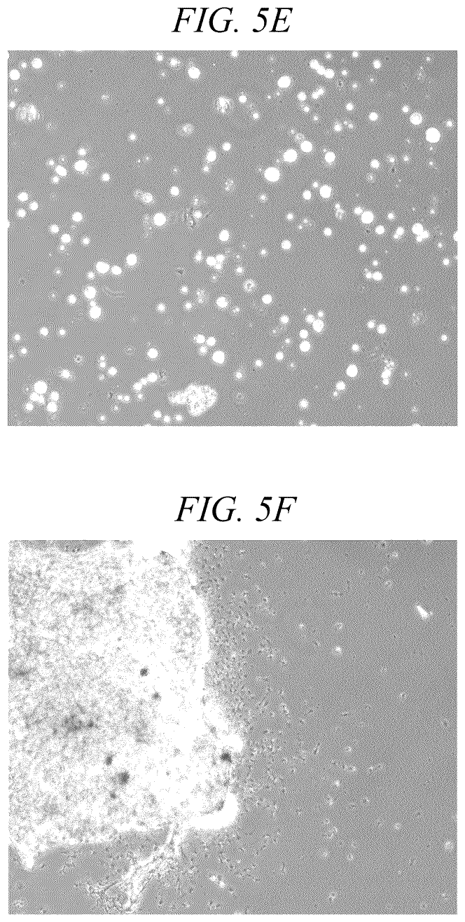

FIG. 5 is an enlarged photograph of primary cells taken under a microscope (magnification: 100×, product name: AE31E, manufacturer: Motic company) on the 1st day (gill, hepatopancreas, heart) and 9th day (muscle) after seeding, which were proliferating from the muscle (a, b), gill (c, d), hepatopancreas (e) and heart (f) tissue of Litopenaeus vannamei according to the present invention.

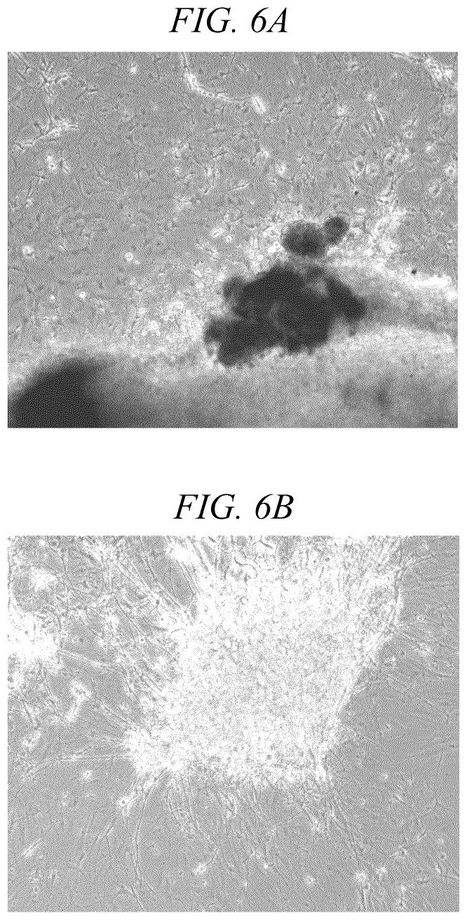

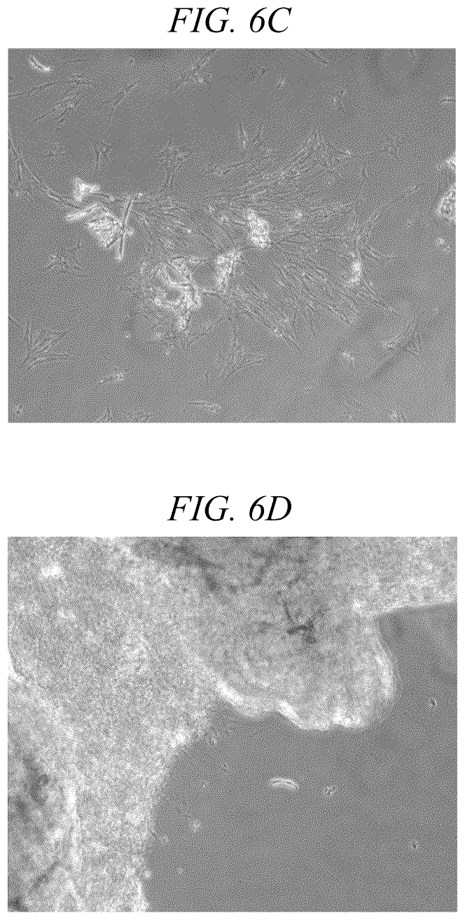

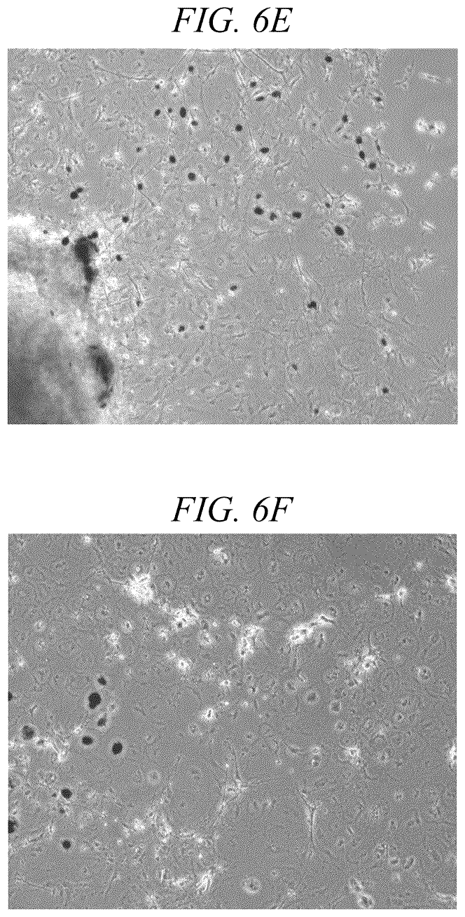

FIG. 6 is an enlarged photograph of primary cells taken under a microscope (magnification: 100×, product name: AE31E, manufacturer: Motic company) on the 8th day (muscle, skin) and 11th day (heart) after seeding, which were proliferating from the muscle (a, b), heart (c, d), and skin (e, f) tissue of Todarodes pacificus according to the present invention.

According to FIGS. 4 to 6, it was observed that primary cells from the tissue were attached to a flask or cell culture dish and proliferated.

### Comparative Example 1

Primary cells were isolated and cultured from aquatic animal tissues in the same manner as in the Example, except that salinity was not adjusted depending on the aquatic animal species and L-15 medium (Sigma-Aldrich company) was used as a basic culture medium.

### Comparative Example 2

Primary cells were isolated and cultured from aquatic animal tissues in the same manner as in the Example, except that the tissues were sectioned using chemical reagents and enzymes such as trypsin or collagenase.

### Comparative Example 3

Primary cells were isolated and cultured from aquatic animal tissues in the same manner as in the Example, except that instead of using a cell culture solution containing complex antibiotics, a cell culture solution containing only penicillin/streptomycin was used.

### [Results of Primary Cell Culture of Aquatic Animal Tissues]

TABLE 3

| | | Comparative | Comparative | Comparative | |
|---|---|---|---|---|---|
| Test | Organism | Example 1 | Example 2 | Example 3 | Example |
| 1^{st} | Todarodes | X | X | X | ◯ |
| | pacificus | | | | |
| | Litopenaeus | X | X | X | ◯ |
| | vannamei | | | | |
| | Anguilla | ◯ | X | X | ◯ |
| | japonica | | | | |
| 2^{nd} | Todarodes | X | X | X | ◯ |
| | pacificus | | | | |
| | Litopenaeus | X | X | X | ◯ |
| | vannamei | | | | |
| | Anguilla | ◯ | ◯ | ◯ | ◯ |
| | japonica | | | | |
| 3^{rd} | Todarodes | X | X | X | ◯ |
| | pacificus | | | | |
| | Litopenaeus | X | X | X | ◯ |
| | vannamei | | | | |
| | Anguilla | ◯ | ◯ | X | ◯ |
| | japonica | | | | |

* ◯: Successfully acquired primary cells, X: Failed to acquire primary cells

As can be seen in Table 3 above, in Comparative Example 1, if the appropriate salinity in the cell culture solution was not maintained depending on the aquatic animal species, tissue and cell survival was impossible due to an imbalance in osmotic pressure inside and outside the cells. In the case of Todarodes pacificus and Litopenaeus vannamei used in the present invention, cell acquisition failed in all experiments. In Comparative Example 2, as a result of using chemical reagents, invertebrates (Litopenaeus vannamei, Todarodes pacificus) were sensitive to chemical reagents and failed to acquire primary cells in all experiments. In Comparative Example 3, microorganism contamination occurred in both Todarodes pacificus and Litopenaeus vannamei, and in the case of Anguilla japonica, cells were obtained only once out of a total of three experiments.

Meanwhile, according to the Example, the aquatic animal cell culture showed survival and proliferation effects of aquatic animal tissues and cells without microorganisms and fungal contamination in the water, as can be seen in FIGS. 4 to 6.

As a result, various types of cells obtained by the method presented in the present invention for aquatic animals living in various environments can improve aquatic animal ethical issues by replacing animal testing, and can be used in a wide range of industries, especially these can be used in the food industry to develop cultivated meat & seafood among alternative foods. However, when the present invention is applied to the development of cultivated meat & seafood, it only applies to the isolation and culture stage of primary cells and does not mean that complex antibiotics are used when mass culturing cells for actual cultivated meat & seafood production.

Although the preferred examples of the present invention have been described in detail above, the scope of the present invention is not limited thereto. Various modifications and improvements made by those skilled in the art using the basic concept of the present invention as defined in the following claims also fall within the scope of the present invention.

What is claimed is:

1. A method for isolation and culture of primary cells from aquatic animal tissues, which comprises the following steps of:
isolating tissues from an aquatic animal;
washing and disinfecting the isolated tissues;
physically sectioning the washed and disinfected tissues;
centrifuging the sectioned tissues to isolate cells from a portion of the sectioned tissues; and
culturing the centrifuged tissues and cells with a cell culture solution comprising complex antibiotics,
wherein the complex antibiotics comprises at least two of penicillin/streptomycin, amphotericin B, gentamycin, kanamycin, ampicillin and tetracyclin.

2. The method according to claim 1, wherein the complex antibiotics comprises penicillin/streptomycin, amphotericin B, gentamycin, kanamycin, ampicillin and tetracyclin.

3. The method according to claim 1, wherein the culturing step comprises a first culture with a cell culture solution comprising the complex antibiotics and then a second culture with a cell culture solution comprising a single antibiotic.

4. The method according to claim 3, wherein the single antibiotic is one of penicillin/streptomycin, amphotericin B, gentamycin, kanamycin, ampicillin and tetracyclin.

5. The method according to claim 3, wherein the first culture is cultured for a period of 10 to 20 days, and
the second culture is cultured for a period of 5 days or more and 15 days or less.

6. The method according to claim 3, wherein at least one of the cell culture solution comprising the complex antibiotics and the cell culture solution comprising the single antibiotic comprises sea salt.

7. The method according to claim 3, wherein the salt concentration of at least one of the cell culture solution comprising the complex antibiotics and the cell culture solution comprising the single antibiotic is greater than 0 ppt and less than or equal to 30 ppt.

8. The method according to claim 1, wherein the washing and disinfecting step involves removing microorganisms by disinfecting for less than or equal to 1 minute using ethanol.

9. The method according to claim 1, wherein the section is cut using one or more types of scissors, scalpel and tweezers.

10. The method according to claim 1, wherein the culturing step is performed under a temperature condition of 10° C. or higher and 30° C. or lower.

11. A primary cell isolated and cultured using the method according to claim 1.


SEQUENCE LISTING (6 sequences)

SEQ ID NO: 1 - DNA, 1593 bp, Anguilla japonica
  source 1..1593: mol_type = genomic DNA
gtggcaatcacccgttgattcttttctactaatcacaaagacattggtaccctatatctagtatttggtgcctgagccggaatagttggaaccgcactaagccttctaatccgtgccgaattaagtcaaccaggcgcccttcttggagacgaccaaatttacaatgtcatcgtcacagcgcatgcctttgtaatgattttctttatagtaataccagtaataattggaggatttggcaactggctcgtgccgttaatgatcggcgccccagacatagcatttccccgaataaacaacataagcttctgacttttaccaccatcattccttcttctgctggcctcctcaggggtagaagctggggccggtacaggttgaaccgtatatcctcctctagctggaaacttagcccatgccggagcatctgttgacctgacaatcttttcacttcaccttgcagggatttcatcaatcctaggggccattaattttattactacaattattaatatgaagccgcctgccattacacagtaccaaaccccactgtttgtatgagctgttttagttaccgctgttctactacttctatccctcccagtcctagctgcaggtattacaatacttctaactgaccgaaatttaaatacaaccttctttgaccctgcagggggtggagacccaatcctctaccaacacctattctgattctttggtcacccagaagtatacattttaatcctaccaggatttggaataatctcacacattgttgcctattattccggtaagaaagaaccatttggatatataggaatagtttgagcaatgatggctatcggacttctaggattcattgtatgagcacaccatatgttcacagtaggaatagacgtagacactcgtgcttacttcacttccgccacaataatcatcgcaattccaaccggagtaaaagtattcagctgactagccacattacacggaggggtcatcaaatgagaaaccccactcctttgagctttgggttttatcttcctatttacagtcgggggcttgacaggtatcgtactagcaaactcatcaatcgacattgtattacatgacacatactatgtagtagctcatttccattatgttctatctataggagcagtctttgctattataggaggctttgtgcactgattccccctgttctcaggctatacactacacagcacatgaaccaaagtacactttggaattatattcgtaggagtaaacctaactttcttcccacaacatttcctagggttagcaggaataccacgacgttattcagactacccagatgcctacaccctgtgaaacacaatctcctctattgggtcactaatttctctcacagccgtaatcctgttcctatttatcctttgagaagcattcactgctaaacgagaagtaaaatgagtagaactcacagaaacaaatgttgaatgactacacggatgtcctccaccataccacacattcgaagaaccagcgtacgtccgagttcaaccgccctcagaagatcaaaaatcagaagccaaagcccacattcaagaaagg

SEQ ID NO: 2 - DNA, 1593 bp, Anguilla japonica
  source 1..1593: mol_type = genomic DNA
gtggcaatcacccgttgattcttttctactaatcacaaagacattggtaccctatatctagtatttggtgcctgagccggaatagttggaaccgcactaagccttctaatccgtgccgaattaagtcaaccaggcgcccttcttggagacgaccaaatttacaatgtcatcgtcacagcgcatgcctttgtaatgattttctttatagtaataccagtaataattggaggatttggcaactggctcgtgccgttaatgatcggcgccccagacatagcatttccccgaataaacaacataagcttctgacttttaccaccatcattccttcttctgctggcctcctcaggggtagaagctggggccggtacaggttgaaccgtatatcctcctctagctggaaacttagcccatgccggagcatctgttgacctgacaatcttttcacttcaccttgcagggatttcatcaatcctaggggccattaattttattactacaattattaatatgaaaccgcctgccattacacagtaccaaaccccactgtttgtatgagctgttttagttaccgctgttctacttcttctatccctgccagtcctagctgcaggtattactatacttctaactgaccgaaatttaaatacaaccttctttgaccctgcagggggtggagacccaatcctctaccaacacctattctgattctttggtcacccagaagtatacattttaatcctaccaggatttggaataatctcacacattgttgcctattattccggtaagaaagaaccatttggatatataggaatagtttgagcaatgatggctatcggacttctaggattcattgtatgagcacaccatatgttcacagtaggaatagacgtagacactcgtgcttacttcacttccgccacaataatcatcgcaattccaaccggagtaaaagtattcagctgactagccacattacacggaggggtcatcaaatgagaaaccccactcctttgagctttaggttttatcttcctatttacagtcgggggcttgacaggtatcgtactagcaaactcatcaatcgacattgtattacatgacacatactatgtagtagctcatttccattatgttctatctataggagcagtctttgctattataggaggctttgtgcactgattccccctgttctcaggctatacactacacagcacatgaaccaaagtacactttggaattatgttcgtaggagtaaacctaactttcttcccacaacatttcctagggttagcaggaataccacgacgttattcagactacccagatgcctacaccctgtgaaacacaatctcctctattgggtcactaatttctctcacagccgtaatcctgttcctatttatcctttgagaagcattcactgctaaacgagaagtaaaatgagtagaactcacagaaacaaatgttgaatgactacacggatgtcctccaccataccacacattcgaagaaccagcgtacgtccgagttcaaccgccctcagaagatcaaaaatcagaagccaaagcccacattcaagaaagg

SEQ ID NO: 3 - DNA, 1533 bp, Litopenaeus vannamei
  source 1..1533: mol_type = genomic DNA
acgcaacgatgattattttctacaaaccacaaagacattggaacattatactttatcttcggggcttgagctggaatagtaggtaccgctcttagacttattatccgagctgaattaggtcaacctgggagcctcattggggatgatcaaatttataacgtagttgtcacagctcacgcttttgtaataattttttttatagttataccaattataattggaggatttggtaattgactagtacctttaatgttaggtgccccagatatagccttccctcggatgaataatataagcttctggttattacctccttctctcacattgcttttatcaagaggaatggttgaaagaggtgtcggaaccggatgaacggtataccctcctttatctgccagtattgctcacgctggagcttcagtagatcttggaattttctctcttcacttagctggagtatcttctattctgggagcagtaaactttataacaactgtaatcaatatacgatctacaggaataactatagaccgtatacctctatttgtatgagcagtatttatcactgctttattactacttttatcattaccagtcttagcaggagctattactatacttttaacagaccgtaatcttaacacatcattcttcgacccagcaggaggaggagacccagttttatatcaacatttattctgattcttcggtcacccataagtatatattttaattttacctgcttttggtataatttcccatattattagtcaagaatctggtaaaaaagaagcattcggaacactcgggataatctatgctatacttgctattggtgtcctaggattcgtagtatgagcacaccatatatttacagtaggtatggatgttgatactcgtgcttattttacatctgcaacaatgattattgccgttcctacaggtattaaaattttcagttgactaggtactcttcatggtactcaacttaactatagtccttctctaatttgagcgctaggatttgtatttctatttactgtgggtggtttaaccggtgttgtactagctaattcttcaattgacattattttacatgatacatactacgtagtagcacatttccactatgttctttcaataggagctgtatttggtatttttgcaggtattgcccactgattccctttatttacggggcttaccttaaaccctaagtgattaaaaattcactttcttgttatattcattggagtaaatattacattcttcccccaacatttcttaggtcttaatggaatacctcgacgatactcagactacccagatgcttattcagcatgaaatgttgtatcttccattggatcaacggtatccctgattgccgtgttaggctttgttataatcgtctgagaagcattaactgctgctcggcctgtagttttttcactattcttaccaacttcaatcgaatgacagcataaccttccaccagcagatcatagttatatagaaattcccttaatcactaat

SEQ ID NO: 4 - DNA, 1533 bp, Litopenaeus vannamei
  source 1..1533: mol_type = genomic DNA
acgcaacgatgattattttctacaaaccacaaagacattggaacattatactttatcttcggggcttgagctggaatagtaggtaccgctcttagacttattatccgagctgaattaggtcaacctgggagcctcattggggatgatcaaatttataacgtagttgtcacagctcacgcttttgtaataattttttttatagttataccaattataattggaggatttggtaattgactagtacctttaatgttaggtgccccagatatagccttccctcgaatgaataatataagcttctggttattacctccttctcttacattgcttttatcaagaggaatggttgaaagaggtgtcggaaccggatgaacggtataccctcctttatctgccagtattgctcacgctggagcttcagtagatcttggaattttctctcttcacttagctggagtatcttctattctgggagcagtaaactttataacaactgtaatcaatatacgatctacaggaataactatagaccgtatacctctatttgtatgagcagtatttatcactgctttattactacttttatcattaccagtcttagcaggagctattactatacttttaacagaccgtaatcttaacacatcattcttcgacccagcaggaggaggagacccagttttatatcaacatttattctgattcttcggtcacccagaagtatatattttaattttacctgcttttggtataatttcccatattattagtcaagaatctggtaaaaaagaagcattcggaacactcgggataatctatgctatacttgctattggtgtcctaggattcgtagtatgagcacaccatatatttacagtaggtatggatgttgatactcgtgcttattttacatctgcaacaatgattattgccgttcctacaggtattaaaattttcagttgactaggtactcttcatggtactcaacttaactatagtccttctctaatttgagcgctaggatttgtatttctatttactgtgggtggtttaaccggtgttgtactagctaattcttcaattgacattattttacatgatacatactacgtagtagcacatttccactatgttctttcaataggagctgtatttggtatttttgcaggtattgctcactgattccctttatttacggggcttaccttaaaccctaagtgattaaaaattcactttcttgttatattcattggagtaaatattacattcttcccccaacatttcttaggtcttaatggaatacctcgacgatactcagactacccagatgcttattcagcatgaaatgttgtatcttccattggatcaacggtatccctgattgccgtgttaggctttgttataatcgtctgagaagcattaactgctgctcggcctgtagttttttcactattcttaccaacttcaatcgaatgacagcataaccttccaccagcagatcatagttatatagaaattcccttaatcactaat

SEQ ID NO: 5 - DNA, 1533 bp, Todarodes pacificus
  source 1..1533: mol_type = genomic DNA
atgcgatgactattttctacaaaccataaagacattggtaccttatattttatctttggtatctgggcaggactattaggtacatcattaagattaatgattcgtaccgaattaggtcaacccggatctttattaaatgatgatcaattatataacgtagtagttactgctcacggattcattataatttttttcatagttatacctattataattggaggatttggtaactggttagttcccttaatattaggtgctccagatatagcattcccacgtataaacaatataagattctgactacttcctccatccttaactcttttattagcttcatctgctgtagaaagaggagccggaacaggttgaacagtttatccccctttatctaggaatttatcccatgctggtccttcagttgatctagcaattttctcactccacttagctggtgtctcttccattttaggtgcaattaatttcattacaactatcttaaatatacgatgagaaggtcttcaaatagaacgtcttcctttatttacatgatctgtatttattacagccattttattgctactctccttaccagtgctagcaggtgcaattactatgctgttaactgatcgaaacttcaatactactttttttgatcctagtggagggggagacccaattttataccaacatttattttgattctttgggcatcccgaagtatatattttaattttacctgcctttggtattatttcacacattgtatctcaccactctttaaagaaagaaatttttggagctttaggtataatttacgccatactatcaattggtctcctaggttttattgtctgagcacatcatatattcaccgtaggaatggatgtagatactcgagcttatttcacatcagcaacaataattattgcaattcctaccggaattaaagtatttagttgattagcaactatttatggttctcctattaaatataatacacctatactctgagcattagggttcatctttttatttactgtaggaggtttaacaggtattattctttctaattcatcattagatattatacttcatgatacatattatgttgtagctcatttccattatgtcttatctataggagccgtgttcgctttatttgctggttttaaccattgataccctttaattacaggattaacactaaaccaacaatgaaccaaagctcattttatgacgatattcttaggtgtcaacgtaactttttttcctcaacattttctaggattagcaggaatacctcgacggtactcagattatccagattgttatactaaatgaaacatagtatcatctataggatctataatttcacttactagtgtgttgttctttatttttattgtttgagaaagtttaatttcacaacgaactgtgatttgatctaaccacataactacatctttagaatgagacaaccgtcttccagttgacttccatagtctccctgaaacaggggctctttatatttaa

SEQ ID NO: 6 - DNA, 1533 bp, Todarodes pacificus
  source 1..1533: mol_type = genomic DNA
atgcgatgactattttctacaaaccataaagacattggtaccttatattttatctttggtatctgggcaggactattaggtacatcattaagattaatgattcgtaccgaattaggtcaacccggatctttattaaatgatgatcaattatataacgtagtagttactgctcacggattcattataatttttttcatagttatacctattataattggaggatttggtaactggttagttcccttaatattaggtgctccagatatagcattcccacgtataaacaatataagattctgactacttcctccatccttaactcttttattagcttcatctgctgtagaaagaggagccggaacaggttgaacagtttatccccctttatctaggaatttatcccatgctggtccttcagttgatctagcaattttctcactccacttagctggtgtctcttccattttaggtgcaattaatttcattacaactatcttaaatatacgatgagaaggtcttcaaatagaacgtcttcctttatttacatgatctgtatttattacagccattttattgctactctccttaccagtgctagcaggtgcaattactatgctgttaactgatcgaaacttcaatactactttttttgatcctagtggagggggagacccaattttataccaacatttattttgattctttgggcatcccgaagtatatattttaattttacctgcctttggtattatttcacacattgtatctcaccactctttaaagaaagaaatttttggagctttaggtataatttacgccatactatcaattggtctcctaggttttattgtctgagcacatcatatattcaccgtaggaatggatgtagatactcgagcttatttcacatcagcaacaataattattgcaattcctaccggaattaaagtatttagttgattagcaactatttatggttctcctattaaatataatacacctatactctgagcattagggttcatctttttatttactgtaggaggtttaacaggtattattctttctaattcatcattagatattatacttcatgatacatattatgttgtagctcatttccattatgtcttatctataggagccgtgttcgctttatttgctggttttaaccattgataccctttaattacaggattaacactaaaccaacaatgaaccaaagcccattttatgacgatattcttaggtgtcaacgtaactttttttcctcaacattttctaggattagcaggaatacctcgacggtactcagattatccagattgttatactaaatgaaacatagtatcatctataggatctataatttcacttactagtgtgttgttctttatttttattgtttgagaaagtttaatttcacaacgaactgtgatttgatctaaccacataactacatctttagaatgagacaaccgtcttccagttgacttccatagtctccctgaaacaggggctctttatatttaa